(19) United States
(12) Patent Application Publication
(10) Pub. No.: US 2023/0070992 A1
(43) Pub. Date: Mar. 9, 2023

(54) METHOD FOR POLYGENIC RISK EVALUATION

(71) Applicant: Ever Fortune.AI CO., Ltd., Taichung City (TW)

(72) Inventors: Yu-Cheng Lee, Taichung City (TW); Chien-Hao Huang, Taichung City (TW)

(21) Appl. No.: 17/500,035

(22) Filed: Oct. 13, 2021

(30) Foreign Application Priority Data
Aug. 27, 2021 (TW) 110131868

(51) Int. Cl. G16H 50/30; G16H 50/20; G16B 30/00; G16B 20/00

(52) U.S. Cl. CPC G16H 50/30; G16H 50/20; G16B 30/00; G16B 20/00; G06F 17/18

(57) ABSTRACT

The present invention relates to genetic risk assessment system and the method using programmable logic gate array (FPGA) and accelerator card by computing the frequency of the multiple gene detection sites and multiple disease prevalence rates, and to include steps for generating the result in the display.

### CROSS-REFERENCE TO RELATED APPLICATIONS

The present application is based on, and claims priority from, Taiwan Patent Application Serial Number 110131868, filed Aug. 27, 2021, the disclosure of which is hereby incorporated by reference herein in its entirety.

### TECHNICAL FIELD

The invention relates to a risk determination system of gene detection, which uses a field programmable gate array (FPGA) and an accelerator card to calculate the results of polygenic loci through an algorithm based on gene detection data and body constitution diagnosis data.

### BACKGROUND

In genetic epidemiology, gene and environment are two major aspects that mainly affect human diseases. How to evaluate the physiological response of users is uncertain. Referring to the patent No. M606684 of Republic of China (hereinafter referred to as the '684), titled “personalized specific metabolic gene nutritional supplement pairing device”, it discloses that the disclosure of using personalized specific metabolic gene nutritional supplement pairing can help clinicians accurately use various gene sequences in the blood glucose metabolic pathway and cell message transmission pathway to provide prescriptions that combine drug sensitive genes and enhance nutritional efficacy. By detecting 16 specific single nucleotide polymorphism (SNP) loci, 13 critical gene loci and four cell levels are classified, and one of the different metabolic pathways is evaluated and analyzed through an algorithm. The method disclosed in the '684 patent sequentially comprises a gene sequencing module; a single gene risk generation module; a polygenic metabolic comprehensive risk evaluation module, which includes a comprehensive four types of risk calculating unit, a database unit and a sorting unit to calculate the risk index of the final blood glucose metabolic pathway; and a display module for displaying the molecular level risk report form and chart of blood glucose metabolism for clinicians to issue targeted nutrition prescriptions. The database used in the '684 patent contains only 13 gene loci. The incidence rate of these loci leads to the probability of certain human characteristics, which belongs to simple analysis.

In another previous art, titled “machine learning disease prediction and treatment prioritization” of U.S. patent Publication No. 20210104321A1 (hereinafter referred to as 321'), it discloses that machine learning is applied to identify one or more records with a specific phenotype. The disclosure in the '321 patent includes receiving a plurality of first records; receiving a plurality of second records; applying a machine learning algorithm to at least one first record and at least one second record to determine a classifier; and applying the classifier to the third records to identify one or more third records associated with the specific phenotype. The design premise of this application is that there are endless memory resources or logic gates available, which can not reduce the usage cost of mobile devices in application materials.

In yet another previous art, titled “system and method for delivering polygenic-based predictions of complex traits and risks” of U.S. patent Publication No. 20210118571A1 (hereinafter referred to as 571'), it discloses that the referred to as '571), the polygenic disease risk score is further calculated based on the eMERGE genome data provided by the National Human Genome Institute and the patient's age and gender. The '571 patent disclosure does not provide a visual system function, and it is not very available to the majority of the Chinese population for risk prediction.

### SUMMARY

The problem to be solved by the invention includes calculating the risk value by combining a plurality of gene detection loci (sites) and a plurality of disease prevalence rates in Taiwan; fast detecting operation through a single FPGA or by the FPGA combining with an accelerator card; achieving consistency between the result of detecting operation and the operation of a server to generate an alert output, so as to improve the prediction accuracy and display it on the display.

The method for solving the problem of the invention includes applying the method of the invention to the Taiwan Han Chinese cell and Genome Bank, which can further make the prediction results applicable to the health risk assessment required by the Han people in Taiwan. Compared with the '684 patent, the present invention performs a cumulative estimation for the occurrence frequency of multiple gene detection points to the prevalence rate, and can determine whether to affect one or more human characteristics through more gene loci.

The method of the invention is also applied to perform a rapid detecting operation by an FPGA or by the FPGA combined with an accelerator card, plan the input reading signal and the controller in the FPGA respectively, and combine the average value and standard deviation of repeated operation into the circuit of the accelerator card, which can simplify the realization of hardware resources necessary for risk value of polygenic detection.

By comparing, the invention applying the microprocessor of ARM to couple gene analyzers of different brands can accelerate data analysis to increase the processing efficiency by 2˜3 times, and the power consumption efficiency can be increased by 30%˜200%. Deducting the time of heat engine and setting test tube, the time of operation and analysis that may take more than three hours for a gene locus can be shortened to 30 minutes to obtain the results. It can significantly save energy and reduce operating costs.

A method for polygenic risk evaluation comprises the following steps: reading a gene sequencing output signal of a user by a gene detection device and transmitting said gene sequencing output signal to a field programmable logic gate array (FPGA); transmitting a questionnaire result of said user to the field programmable logic gate array (FPGA) through a reading device; accelerating an operation through a built-in genome data of an acceleration card; determining a mean value and standard deviation based on the questionnaire result and a prevalence of a disease; performing a risk prediction through a supervised machine learning algorithm and a plurality of classifiers by a server; outputting results of the risk prediction to a display device; and classifying a health risk level by a grading and a critical value marking based the results of the risk prediction.

The method further comprises applying a binary search method and a recursive process to reduce a complexity of array value search and calculation of said field programmable logic gate array. The gene detection device and the reading device are electrically couple the server through RJ45, D-sub, USB, GPIO, SPI or CCI for data integration. The reading step reads a genome dataset inside a gene sequencer, including diseases and high-density detection loci generated by corresponding bases of the diseases. The method further comprises selecting and comparing said built-in genome data by a data generator. The determining step includes a principal component analysis which is applying a covariance matrix to determine that a sum of the first five principal components or a variation percentage of principal components exceeds a pre-determined percentage of cumulative proportion of an original data. The performing a risk prediction performs a test data prediction after being trained by a training unit. The results of risk prediction are presented in scree plot, heat plot or MDS plot, and the critical value is displayed by different colors of points and lines.

### BRIEF DESCRIPTION OF THE DRAWINGS

FIG. 1 shows a block diagram of a health auxiliary identification system of the invention.

FIG. 2 shows a probability map of risk of polygene detection for diabetes mellitus of the invention.

FIG. 3 shows a high risk threshold of polygene detection for diabetes mellitus of the invention.

FIG. 4 shows a flow chart of the proposed method of the invention.

FIG. 5 shows a slope diagram and a risk prediction diagram of the cancer occurrence prediction of the invention.

FIG. 6 shows a multidimensional scale diagram of cancer occurrence prediction of the invention.

FIG. 7 shows a hot zone diagram of cancer occurrence prediction of the invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

The risk identification system of gene detection of the present invention is described in detail below with reference to the accompanying drawings.

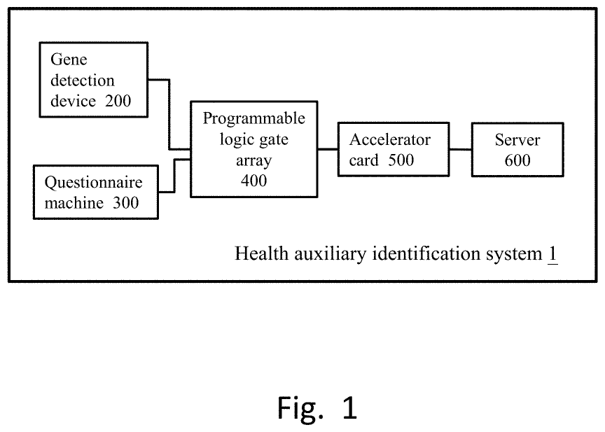

FIG. 1 shows a block diagram of a, including a gene detection device 200 electrically coupled to a programmable logic gate array 400 through USB 2.0 for transmitting signals; a questionnaire machine 300 electrically coupled to the programmable logic gate array 400 through a D-sub interface to transmit signals; a hardware acceleration card 500 electrically coupled to the programmable logic gate array 400 through a CCI interface to transmit signals; a server 600 electrically coupled to the hardware accelerator card 500 through USB 2.0. When the programmable logic gate array 400 and the hardware accelerator card 500 perform operation, another operation process can be performed at the same time. The programmable logic gate array 400 can be used for I/O interface design, depending on its brand (such as Stratix 10, REFLEX CES XpressVUP-LP9P, Arria 10 GX FPGA), specification (such as RJ45, D-sub, USB, GPIO, SPI, CCI) and the number of ports, it can be used for communication protocol or data line control on the signal line.

In the health assisted identification system of FIG. 1, the server can be electrically connected for the purpose of troubleshooting during test design.

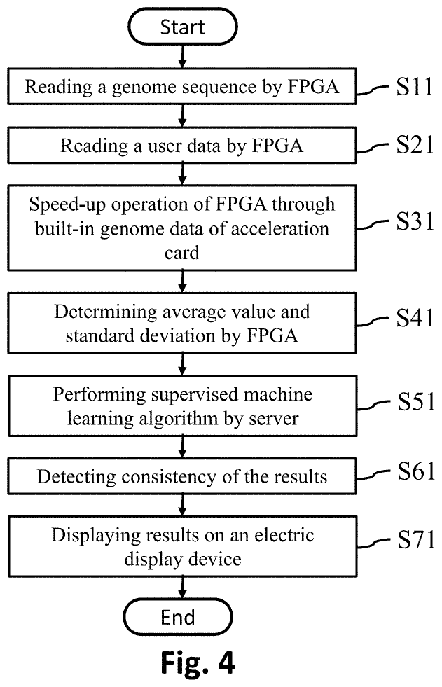

The field programmed gate array (FPGA) in FIG. 1 can be an Altera Cyclone V 28 nm FPGA. The field programmed gate array (FPGA) will perform a gene sequencing reading in step S11 and a user data input in step S21, shown in FIG. 4. In the gene sequencing reading of the step S11, the USB 2.0 interface of the programmable logic gate array 400 reads a gene sequencing output signal of the gene detection device 200 for the user using. In the user data input of the step S21, the data from the questionnaire machine 300 or other electronic questionnaires is read by the D-sub interface of the programmable logic gate array 400.

In addition, the steps to be performed by the programmable logic gate array 400 are shown in FIG. 4. The programmable logic gate array 400 is connected to an acceleration card 500 through a CCL interface to execute a data Speed-up calculation in step S31. The data Speed-up calculation of the step S31 adopts the Aria 10 GX FPGA special acceleration card developed by Intel for big data, which is compatible with Apache Hadoop and Apache Spark systems, but not limited to the acceleration card of this brand, as long as it is compatible with the signals or databases of Affymetrix, Agilent, Illumina and other machines. The acceleration card is similar to the host of Nintendo and assists for speed-up operation of card clip single chip of acceleration FPGA. The programmed logic gate array 400 performs an algorithm operation in step S41 after retrieving one of the preprocessed gene bank data from the accelerator card 500. The preprocessing refers to data processing such as compression, classification and search of gene database. In addition, the algorithm operation in the step S41 can be that when performing secondary data analysis of next-generation gene sequencing, the average value can be calculated through the development board of the programmed logic gate array, and the standard deviation can be calculated through an accelerator card, or the standard deviation can be calculated through the development board of the programmed logic gate array, and the average value can be calculated through the accelerator card, which are parallel processing operation. The algorithm of the average value of risk (Ave) is defined as Formula (1), wherein A is the risk score, F is the occurrence frequency, and the average value is equal to the sum of a single item (the occurrence frequency of each gene locus multiplying the risk value).

Σavg=A1×F1+A2×F2+A3×F3+ . . . (1)

The standard deviation can be defined as Formula (2).

$$( 2 )$$

$$\sqrt{{( A 1 )}^{2} \times F 1 \times ( 1 - F 1 ) + {( A 2 )}^{2} \times F 2 \times ( 1 - F 2 ) + {( A 3 )}^{2} \times F 3 \times ( 1 - F 3 ) + ⋯}$$

When we load the burn-in program of the programmable logic gate array development board into different compatible library through the RTL (Register Transistor Level) simulator to conduct analysis report, we can obtain the values in Table 1 by comparison.

TABLE 1
Comparison of gene detection device and programmed logic
gate array for executive performance of single gene locus

| Brand of gene | | Required executive time |
|---|---|---|
| detection device | Altera Cyclone V FPGA | for single gene locus |
| (Manufacturer) | (Gate Counts) | (instruction/clock) |
| Affymetrix | 44K+ | 30M+ |
| Agilent | 52K+ | 44M+ |
| Illumina | 44K+ | 88M+ |

It can be seen from the table 1 that the number of microprocessors and lines in gene detection devices (analyzers) of different brands may lead to differences in the performance of coupled programmed logic gate arrays. Especially in the implementation of gene detection, the alignment of gene sequences is very time-consuming. Therefore, the gene detection device of a better manufacturer can be appropriately selected as the consideration for obtaining fast operation. For example, Illumina has the highest executive efficiency.

Gene Detection Device

Human chromosomes are composed of proteins and genes, and the genes consist of four nucleic acid bases; they are adenine (A), cytosine (C), guanine (G), and thymine (T). These nucleic acid bases in DNA could be formed in different combination and a lengthy arrangement or known as a sequence. The order of sequence with these four bases determines the factors in the human genetic codes for human diseases, growth conditions, aging conditions, and so on. In addition, the possibility of two or more nucleotides existing at a specific and localized locus (site) of the genome composed of A, T, C and G will lead to variation of gene deletion, insertion or substitution. In genetics, if the occurrence frequency of these specific and localized loci is less than or equal to 1% of the corresponding allele, it is called mutation. Therefore, the difference between specific and localized loci (SNP) and mutation is greater than 1% for the former and equal to or less than 1% for the latter. Although the proportion of mutation is small, the consumption required for overall hardware is very large. Therefore, the selection of specific hardware, such as Illumina, can shorten the time to achieve the effectiveness of saving time, power and manpower for commercial purpose.

In recent years, the latest progress in machine learning analysis of large genome data sets has made it possible to create polygenic predictors of complex human characteristics, including the risk of many important and complex diseases, which are usually affected by many genetic variations. Each variation has little impact on the overall risk, but in the polygenic risk predictor, the lifetime (or age range) risk of disease is determined through a numerical function captured by score, wherein the score depends on the status of thousands of individual genetic variations (i.e. single nucleotide polymorphism (SNP)). Therefore, polygenic scoring method has also become one of the applications of machine learning.

Polygenic Scoring Method

Gene environment interaction plays an important role in genetic traits and has attracted more and more attention in genetic epidemiology. The detection of gene environment interaction through genome-wide association research can integrate the interaction effects of mononucleotide polymorphism and environmental factors into one test, so as to improve our understanding of the causes of disease, such as risk ranking, assisting clinical diagnosis, testing characteristics with gene overlap (such as depression, measuring cardiovascular disease), inserting the missing characteristics and personalized treatment,

Algorithm of weight of corresponding gene risk value

Polygenic risk score (PRS) is sum of the effective size β1SNP1, plus β2SNP2 . . . added to βnSNPn, as shown in the formula 3.

PRS=β1SNP1+β2SNP2+βnSNPn (3)

Wherein β is the effective size, SNP is the pairs of risk genes, and n is the number of SNP.

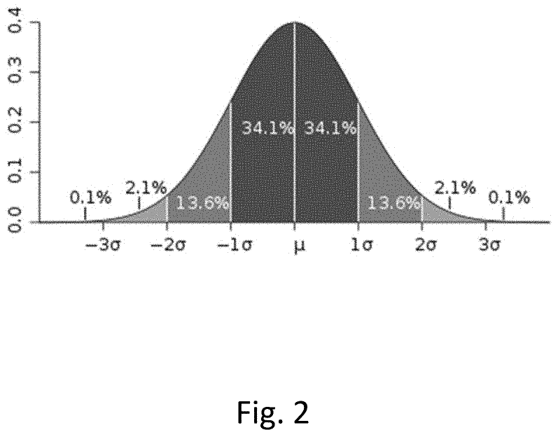

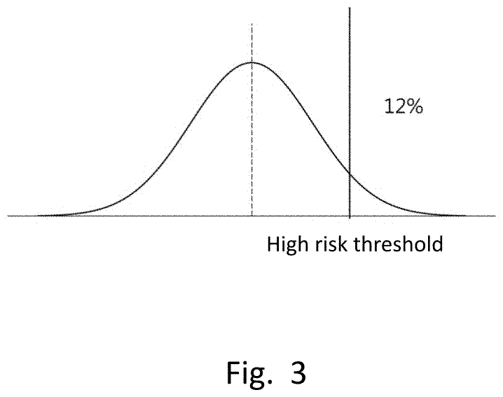

FIG. 2 shows a general risk distribution function which is a Gaussian distribution, wherein the horizontal axis is log (logarithm) risk score and the vertical axis is population. We can also use the probability function of a specific disease obtained by Bayesian theory as the occurrence risk coefficient. For the purpose of disease prediction, the variance of the distribution function is the key to determine the stratification or risk discrimination. For example, compared with FIG. 2, the distribution of a disease is more people or higher proportion of population toward the right-side of the horizontal axis. In this model, the population can be the number of cases or cases of a disease. By overlapping the general risk distribution function with the allocated population of the disease, we can know what the risk threshold is. As shown in FIG. 3, taking diabetes as an example, the prevalence of diabetes in Taiwan is about 12%, and 88% (100%−12%) value can be calculated by means of the mean average and standard deviation. When genetic testing is performed, the cumulative value of the results exceeds the high-risk threshold, it is determined as high-risk.

Generally, the performance of corresponding gene risk value depends on inheritability, effective size and sample size. The most ideal inheritability refers to the real correlation coefficient, which does not need to be estimated or will not produce selection error. However, a scientifically acceptable way is to adopt a specific gene platform, such as GWAS Catalog database platform, through which the maximum potential value related to the variance is determined.

In the GWAS study, the main analysis method of locus search is linkage disequilibrium (LD) analysis. Alleles at different loci appear with a certain frequency in the population, but in a population, if the frequency of two alleles at different loci shown on the same chromosome is higher than the expected random frequency, it is called linkage disequilibrium. By detecting a large number of genetic marker loci throughout the genome, or genetic markers near candidate genes, disease-related loci can be found. In addition, too few samples can easily lead to false positive correlation, but this problem can be improved by comparing with the public GWAS database, or further using more databases to verify the correctness of SNP screening through big data. At present, the existing tools include C+T, PLINK, PRSice2, bigsnpR, LDpred2, SBayersR, Lassosum, PRS-CS, JAMPred, etc., which can be used for regression calculation required by clumping phenotype.

Cancer as an Example

For Hereditary breast and ovarian cancer syndrome, the two most important genes contributing to this syndrome are BRCA1 and BRCA2 which were discovered in the United States in the 1990. In 1990, Hall et al. studied early-onset and hereditary breast cancer families. Through linkage analysis, they found that chromosome 17q21 was highly correlated with early-onset familial breast cancer. Later, in 1994, Miki et al. identified that the BRCA1 gene on chromosome 17q21 is the gene causing breast cancer (and ovarian cancer). In the same year, Wooster et al. found that 13q12-13 was also associated with breast cancer, so they found BRCA2 gene. BRCA1 has 24 exons, and the translated BRCA1 protein has 1863 amino acids. BRCA2 has 27 exons, and the translated BRCA2 protein has 3418 amino acids. These two genes belong to tumor suppressor genes, which are responsible for the repair mechanism of double stranded DNA damage. When the double stranded DNA in the cell is damaged, the cell has two ways to repair. The first repair method is called homologous recombination and the other is non homologous end-joint. Only through homologous recombination, the double stranded DNA can be repaired correctly. The double stranded DNA repair mechanism involved in BRCA1 and BRCA2 is homologous recombination. Therefore, if one of these two genes is defective, the double stranded DNA will not be repaired correctly after being attacked and broken. When the DNA damage in the cell accumulates to a certain extent, the cell will become cancerous. There are many proteins, main Fanconi's pathway related proteins, involved in homologous recombination repair. In recent research, it has found that pathogenic variation of genes involved in homologous recombination will also occur phenotypes similar to BRCA1 and BRCA2 mutations, that is, breast cancer, ovarian cancer or related cancer.

Therefore, if you want to further understand the occurrence of breast cancer, ovarian cancer or related cancer, you can know the prevalence of which cancer through the statistical PRS table. Referring to the table 2, we can obtain the SNP values of different cancers through a statistical software by using its internal algorithm. Taking breast cancer as an example, there are 4530 SNP groups that may affect to disease, accompanied by 1052 possible standard deviations. The polygenic risk score due to heredity has the incidence rate with weight of 0.77, accompanied by standard deviation of 0.04. The polygenic risk score of area related under the curve was 0.73, accompanied by a standard deviation of 0.01. The area under the curve (AUC) is calculated as the formula 4.

AUC=Φsqrt(h\hat{ }2/2) (4)

Wherein Φ is the cumulative density function of standard normal distribution. When we know the number of diseases, we can classify cancers with other weight factors.

TABLE 2
Estimation of morbidity variation and heritability of various cancers

| | | Estimated number | Heritability: | Optimal PRS |
|---|---|---|---|---|
| | | of independent | morbidity of optimal | related to area |
| Occurrence | | diseases (standard | PRS (standard | under curve |
| number | cancers | deviation) | deviation) | (AUC) |
| Less than | Chronic | 2025(1501) | 1.62(0.37) | 0.82(0.03) |
| 10000 | lymphoma | | | |
| | Esophageal | 2642(2515) | 1.24(0.36) | 0.78(0.03) |
| | cancer | | | |
| | Testicular | 2598(2088) | 2.81(0.40) | 0.88(0.02) |
| | cancer | | | |
| | Oropharyngeal | 3623(2060) | 0.68(0.27) | 0.72(0.04) |
| | cancer | | | |
| | Pancreatic | 1757(1490) | 0.60(0.16) | 0.71(0.03) |
| | cancer | | | |
| 10000 to | Kidney cancer | 2220(1555) | 0.57(0.12) | 0.70(0.02) |
| 25000 | Brain cancer | 2364(1593) | 0.87(0.11) | 0.75(0.01) |
| | Melanoma | 484(173) | 0.57(0.08) | 0.70(0.01) |
| | Colorectal | 1484(696) | 0.43(0.10) | 0.68(0.02) |
| | cancer | | | |
| | Skin cancer | 1052(772) | 0.27(0.07) | 0.64(0.02) |
| | Ovarian | 1015(715) | 0.24(0.06) | 0.64(0.02) |
| | cancer | | | |
| More than | Prostate | 6096(2750) | 0.39(0.06) | 0.67(0.01) |
| 25000 | cancer | | | |
| | Breast cancer | 4530(1052) | 0.77(0.04) | 0.73(0.01) |
| | Lung cancer | 7599(1615) | 0.60(0.03) | 0.71(0.01) |

The above examples of cancers are only one of the embodiments to which the invention can be applied, but are not limited to cancers. Similarly, the polygenic risk score (PRS) can be applied to other rare diseases or test items.

Binary Search Method and Application of Recursive Function

The invention applied the Taiwan Han Chinese cell and Genome bank, and expands the genome bank into a 1×N array, and stores it in an acceleration card, so as to facilitate the comparison of different gene sequences. Because the array may be too long, it must be simplified operation by different sorting methods. The algorithm of a binary search method used in the invention is shown in Table 3.

TABLE 3
Algorithm of binary search method and use of recursive function

Binarysearch(A,n,x)

| | |
|---|---|
| {start<−0 | /*first localized locus*/ |
| end<−n−1 | /*last localized locus*/ |

while (start<=end)
{

| | |
|---|---|
| mid<−(start+end)/2} | /*Start searching in the middle*/ |
| If A[mid]==x | /*If the value found in the middle |
| | is what we need, stop*/ |

return mid

| | |
|---|---|
| elseif x<A[mid] | /*If the value found in the middle |

```
is not what we need, the recursive function is executed and shorten the
search interval*/
        {
          end<−mid−1
        }
        else
        {
          Start<−mid+1
        }
    }
} return−1
```

The above algorithm is applied to a known polygenic risk score to divide the sequence in the searched genome bank into two segments in to search the gene sequence we want to compare. In addition, we can also apply the FORK function, and then apply this algorithm to perform multi-stage parallel processing operations to speed up the search for pairing.

Array Transposition Algorithm

In addition, when converting the array of the genome bank into 1×N array, A1 [1] to A1 [n], we also need to arrange it in reverse order into another array A1 [n] to A1 [1], and apply the following program to search and compare the loci in the database in the accelerator card.

TABLE 4
Array transposition algorithm

| | |
|---|---|
| start=0 | /* first localized locus */ |
| end=n−1 | /* last localized locus */ |
| While start<end | /*When the previous |

localized locus is after the last localized locus, execute the following
SWAP function
{

| | |
|---|---|
| swap(array[start],array[end]; | /* The values of the two |
| | localized locus are interchanged */ |

```
  start=start+1;
  end=end−1
}
```

The gene sequence to be searched can be directly front-back interchanged without adding a temporary blank array by the algorithm of array transposition. If we want to find a gene sequence with AATTCCGG, as GGCCTTAA appears in the genome bank, it is also an effective gene sequence, so we must apply the above algorithm. The complexity of this algorithm is lower than that of the general temp, and it can also save the computing time of the processor.

In addition, the operation step S41 of the algorithm can also be factor analysis, principal component analysis. When the factor analysis is used, it is applicable to the existence of unique factor, such as rare diseases. When the principal component analysis is used, the contribution of polygenic loci between each other for a feature is considered, and not every factor is included. The application of the invention is applying the covariance matrix to determine that the sum of the first five principal components or the variation percentage of the principal components exceeds 99% of the cumulative proportion of the original data.

Supervised Machine Learning

The server 600 in FIG. 1 can debug the programmable logic gate array 400 through machine learning operation, and in the verification of detection result of the step S61 compare the consistency of the results of the algorithm operation in the step 41 and the server debugging in the step 51.

In the server debugging step S51, the server performs risk prediction through a supervised machine learning algorithm and several classifier modules.

Another algorithm used is the supervised random forest algorithm in machine learning, which mainly depends on a large number of genome bank data and performs training. Supervised machine learning can be divided into classification and regression algorithms. In the verification of detection result of the step S61 of the invention, the consistency judgment is made in the classification method and regression method, so the use of random forest also becomes a verification method to judge whether the performance of the threshold value of the risk interval is correct.

In the process of classification, we do not want to produce overfitting in the operation, that is, the probability distribution is too close or accurately matches the specific gene data sequence, so that we can not well adjust other data or predict the future observation results. Therefore, using multiple decision trees for classification can also produce the advantage of shortening the operation time of processor. The second advantage is that the random forest method can achieve a high degree of accurate prediction, especially in a large number of databases. The third advantage is that for the lack of certain values, especially when gene pairs may not be significant enough for some diseases, they can be estimated. For example, decision tree A will produce output result of GENO1, decision tree B will produce output result GENO2, and decision tree C will produce result GENO1; when all decision trees are placed together like a forest, we can know that the generation of GENO1 and GENO2 is 2:1, so the prediction result is GENO1.

The application of random forest algorithm and the operation of the server can reduce the order of the entropy of a random change in the gene sequence into a lower random change. When we want to obtain information gain, it can subtract the higher-order entropy value from the lower-order entropy value for classification, such as occurrence frequency and prevalence. Because the proposed invention uses the probability of correlation measurement as the classification of nodes, it can obtain multiple decision nodes through the classification algorithm; In the invention, after loading the gene database as the dataset, we can select Bayes classifier, Panda classifier, Numpy classifier, etc., and check the decision points one by one we want to meet the conditions, but not limited to the above classifiers. After classification, we will divide it into two data frames, which are set as a training unit and a test unit. Then, the selected classifier performs matrix factorization or tensor factorization for the training unit, through the initialization of random state and the conditional times of execution, a random forest classifier is established. Next, the trained classifier is applied to the test unit, and then its features are observed. Finally, through the comparison between FPGA and the accelerator card by operation results of the server, we can further reduce the occurrence of false positive and provide correct results of risk prediction.

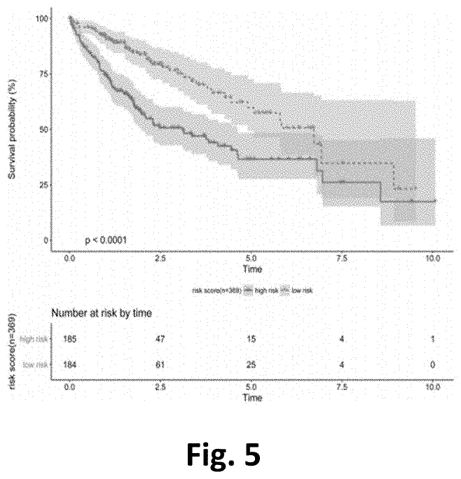

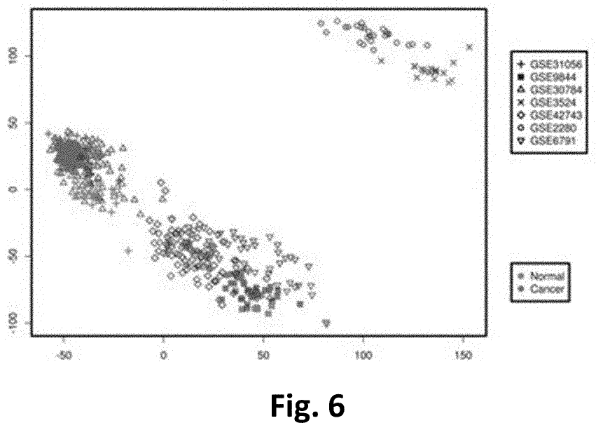

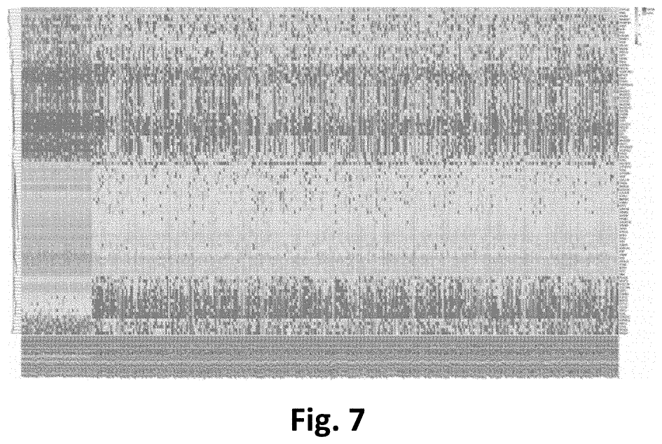

The results of risk prediction are presented in scree plot, heat plot, or MDS plot as shown in FIG. 5 to FIG. 7 through a result output step S71 which displays an electric display device.

As will be understood by persons skilled in the art, the foregoing preferred embodiment of the present invention illustrates the present invention rather than limiting the present invention. Having described the invention in connection with a preferred embodiment, modifications will be suggested to those skilled in the art. Thus, the invention is not to be limited to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. A method for polygenic risk evaluation, comprising:
reading a gene sequencing output signal of a user by a gene detection device and transmitting said gene sequencing output signal to a field programmable logic gate array (FPGA);
transmitting a questionnaire result of said user to said field programmable logic gate array (FPGA) through a reading device;
accelerating an operation through a built-in genome data of an acceleration card;
determining a mean value and standard deviation based on said questionnaire result and a prevalence of a disease; and
performing a risk prediction through a supervised machine learning algorithm and a plurality of classifiers by a server.

2. The method of claim 1, wherein said field programmable logic gate array (FPGA) is coupled to said acceleration card.

3. The method of claim 1, further comprising applying a binary search method and a recursive process to reduce a complexity of array value search and calculation of said field programmable logic gate array.

4. The method of claim 1, wherein said gene detection device and said reading device are electrically couple said server through RJ45, D-sub, USB, GPIO, SPI or CCI for data integration.

5. The method of claim 1, wherein said reading step reads a genome dataset inside a gene sequencer, including diseases and high-density detection loci generated by corresponding bases of said diseases and.

6. The method of claim 5, further comprising selecting and comparing said built-in genome data by a data generator.

7. The method of claim 1, wherein said determining step includes a principal component analysis.

8. The method of claim 7, wherein said principal component analysis is applying a covariance matrix to determine that a sum of the first five principal components or a variation percentage of principal components exceeds a pre-determined percentage of cumulative proportion of an original data.

9. The method of claim 1, wherein said performing a risk prediction performs a test data prediction after being trained by a training unit.

11. A method for polygenic risk evaluation, comprising:
reading a gene sequencing output signal of a user by a gene detection device and transmitting said gene sequencing output signal to a field programmable logic gate array (FPGA);
transmitting a questionnaire result of said user to said field programmable logic gate array (FPGA) through a reading device;
accelerating an operation through a built-in genome data of an acceleration card;
determining a mean value and standard deviation based on said questionnaire result and a prevalence of a disease; and
performing a risk prediction through a supervised machine learning algorithm and a plurality of classifiers by a server;
outputting results of said risk prediction to a display device; and
classifying a health risk level by a grading and a critical value marking based said results of the risk prediction.

11. The method of claim 9, wherein said field programmable logic gate array (FPGA) is coupled to said acceleration card.

12. The method of claim 9, further comprising applying a binary search method and a recursive process to reduce a complexity of array value search and calculation of said field programmable logic gate array.

13. The method of claim 9, wherein said gene detection device and said reading device are electrically couple said server through RJ45, D-sub, USB, GPIO, SPI or CCI for data integration.

14. The method of claim 9, wherein said reading step reads a genome dataset inside a gene sequencer, including diseases and high-density detection loci generated by corresponding bases of said diseases.

15. The method of claim 14, further comprising selecting and comparing said built-in genome data by a data generator.

16. The method of claim 9, wherein said determining step includes a principal component analysis.

17. The method of claim 16, wherein said principal component analysis is applying a covariance matrix to determine that a sum of the first five principal components or a variation percentage of principal components exceeds a pre-determined percentage of cumulative proportion of an original data.

18. The method of claim 9, wherein said performing a risk prediction performs a test data prediction after being trained by a training unit.

19. The method of claim 9, wherein said results of risk prediction are presented in scree plot, heat plot or MDS plot.

20. The method of claim 9, wherein said critical value is displayed by different colors of points and lines.
